(19) United States
(12) Patent Application Publication
(10) Pub. No.: US 2011/0245879 A1
(43) Pub. Date: Oct. 6, 2011

(54) METHOD OF CONNECTING TRANSVERSE BEAM AT TRIANGULAR POSITION OF VERTEBRAL LAMINA

(76) Inventor: Chung-Chun YEH, Taipei City (TW)

(21) Appl. No.: 12/750,942

(22) Filed: Mar. 31, 2010

(51) Int. Cl. A61B 17/88

(52) U.S. Cl. 606/279

(57) ABSTRACT

A method of connecting a transverse beam at a triangular position of a vertebral lamina applied to a position below a spinous process of a vertebral body and above a lamina of vertebral arch comprises the steps of passing a marked position of a lamina of vertebral arch into a transverse hole; passing a transverse rod into the transverse hole and connecting a connecting element at both ends of the transverse rod separately; connecting a vertical rod to a vertical connecting hole of each connecting element; and connecting and fixing another end of the vertical rod to another vertebral body or between vertebral bodies by a fixing element.

### BACKGROUND OF THE INVENTION

1. Field of the Invention

The present invention relates to a method of connecting a transverse beam at a triangular position of a vertebral lamina, and more particularly to a method of installing a transverse beam at a position below a spinous process and above a triangular intersection position of a lamina of vertebral arch of a human spine for connecting and fixing a posterior spine.

2. Description of Related Art

Most conventional methods of connecting and fixing the posterior spine adopt a pedicle screw fixation system (as shown in FIGS. 1a and 1b) that passes a plurality of pedicle screws into pedicle positions of vertebral bodies and fixes the pedicle screws onto the vertebral bodies respectively, and each screw head is connected to a link rod in the direction of a vertebral column for securely connecting each pedicle screw to fix a whole or a portion of a vertebral column to achieve the effect of connecting and fixing the pedicle screws to the vertebral column securely.

However, the conventional methods have been adopted for connecting and fixing a posterior spine for a long time, yet they still have drawbacks and may not be suitable for some treatments.

For instance, the pedicle screw fixation system of this sort is not applicable for certain spinal diseases such as the collapse or osteoporosis of vertebral bodies, because the screw section of a pedicle may not be able to be fixed into the vertebral bodies effectively, and the pedicle screw fixation system of the whole spine may be loosened at a later time, and the involved patient has to take another surgical operation to solve the loosened spine problem.

The foregoing conventional pedicle screw fixation system cannot be used for other connections. If a patient's vertebral column requires another treatment, the pedicle screw fixation system can provide a single vertebral connection and fixation only. For example, an interspinous process fixation device cannot be connected directly and effectively.

Therefore, the aforementioned conventional pedicle screw fixation system and related surgical methods still have their drawbacks and inapplicability, and thus requiring further reviews, improvements and it is necessary to develop an innovative method of connecting a transverse beam at a triangular position of a vertebral lamina and its related device to overcome the problems.

### SUMMARY OF THE INVENTION

Therefore, the present invention overcomes the inapplicability and problems of the conventional pedicle screw fixation system and related surgical methods and provides a simple, effective, stable and applicable spinal fixation to patients, and the method of connecting a transverse beam at a triangular position of a vertebral lamina in accordance with the present invention has an easy-to-operate feature and incurs a low risk.

In the foregoing way of overcoming the problems, apparatuses and tools are implanted into a vertebral body of a patient by the following method to provide a stable spinal fixation.

A transverse hole is transversally formed at a marked position below a spinous process of a vertebral body and above a triangular intersection of a lamina of vertebral arch.

A transverse rod is passed into the transverse hole, and both ends of the transverse rod are coupled separately by a connecting element.

A vertical rod is inserted and connected to a vertical connecting hole of each connecting element.

Another end of the vertical rod is coupled and fixed at another vertebral body or between vertebral bodies by a fixing element.

The marked position of the transverse hole of the lamina of vertebral arch is situated at a vertebral foramen of a bone (a transverse hole is transversally formed at a position of 7˜15 mm from the lamina of vertebral arch on a line connected from the bottom vertex of a lamina of vertebral arch at a curved position to the top vertex of a spinous process).

Therefore, it is a primary objective of the present invention to provide a method of connecting a transverse beam at a triangular position of a vertebral lamina.

Another objective of the present invention is to provide a method of connecting a transverse beam at a triangular position of a vertebral lamina applied for a spinal fixation including devices such as a plurality of transverse rods, a plurality of connecting elements, and a plurality of vertical rods, and a fixing element installed at a marked position below a spinous process of a vertebral body and above a triangular intersection of a lamina of vertebral arch.

A further objective of the present invention is to provide a method of connecting a transverse beam at a triangular position of a vertebral lamina, wherein a transverse hole is formed at a triangular position of 7˜15 mm from the bottom vertex of the lamina of vertebral arch of a bone.

Another further objective of the present invention is to provide a method of connecting a transverse beam at a triangular position of a vertebral lamina, wherein the fixing element is provided for connecting another set of vertical and transverse rods, pedicle screws, vertebral hooks or interspinous process fixation devices.

Therefore, a method of connecting a transverse beam at a triangular position of a vertebral lamina applied to a position below a spinous process of a vertebral body and above a triangular intersection position a lamina of vertebral arch, and the method comprises the following steps:

Pass a marked position of a lamina of vertebral arch of a vertebral body desired to be connected into a transverse hole;

Pass a transverse rod into the transverse hole, and connect a connecting element at both ends of the transverse rod separately;

Connect a vertical rod to a vertical connecting hole of each connecting element; and

Connect and fix another end of the vertical rod at another vertebral body or between vertebral bodies by a fixing element.

In the method of connecting a transverse beam at a triangular position of a vertebral lamina, the marked position of the transverse hole transversally formed at the lamina of vertebral arch is situated at a position of 7˜15 mm from the lamina of vertebral arch on a line connected from a bottom vertex of the lamina of vertebral arch at a curved position to a top vertex of a spinous process.

In the method of connecting a transverse beam at a triangular position of a vertebral lamina, the transverse rod is a transverse rod with an arc degree of curvature.

In the method of connecting a transverse beam at a triangular position of a vertebral lamina, the fixing element is comprised of another transverse rod and a plurality of connecting elements, and the transverse rod is passed into a transverse hole at a marked position of another lamina of vertebral arch, and both ends of the transverse rod are connected by another set of connecting elements and the other set of connecting elements are connected to the vertical rods respectively.

In the method of connecting a transverse beam at a triangular position of a vertebral lamina, the fixing element is an interspinous process fixation device installed between two adjacent spinous processes, and both sides of the interspinous process fixation device are connected to the vertical rods respectively.

In the method of connecting a transverse beam at a triangular position of a vertebral lamina, the fixing element is comprised of a set of pedicle screws implanted and fixed at pedicle positions on both sides of another vertebral body respectively, and the pedicle screws are connected the vertical rods.

In the method of connecting a transverse beam at a triangular position of a vertebral lamina, the fixing element is comprised of a set of spinal hooks hooked to positions on both sides of another vertebral body and connected to the vertical rods.

### BRIEF DESCRIPTION OF THE DRAWINGS

The invention, as well as its many advantages, may be further understood by the following detailed description and drawings in which:

FIGS. 1a and 1b are schematic views of a conventional pedicle screw fixation system;

FIG. 2 is a flow chart of a method of connecting a transverse beam at a triangular position of a vertebral lamina in accordance with the present invention;

FIGS. 3a and 3b are schematic views of a lamina of vertebral arch at a marked position of a vertebral body being transversally passed through a transverse hole in a method of connecting a transverse beam at a triangular position of a vertebral lamina in accordance with the present invention;

FIG. 4 is an exploded view of devices including a transverse rod, a curved transverse rod, a connecting element, and a vertical rod used in a method of connecting a transverse beam at a triangular position of a vertebral lamina in accordance with the present invention;

FIGS. 5a and 5b are schematic views of a method of connecting a transverse beam at a triangular position of a vertebral lamina applied for a vertebral column in accordance with the present invention, wherein FIG. 5b is a representative figure; and

FIG. 6 is a schematic view of a fixing element used in a method of connecting a transverse beam at a triangular position of a vertebral lamina in accordance with the present invention.

### DETAILED DESCRIPTION OF THE INVENTION

The technical characteristics of the present invention will become apparent with the detailed description of preferred embodiments and the illustration of related drawings as follows:

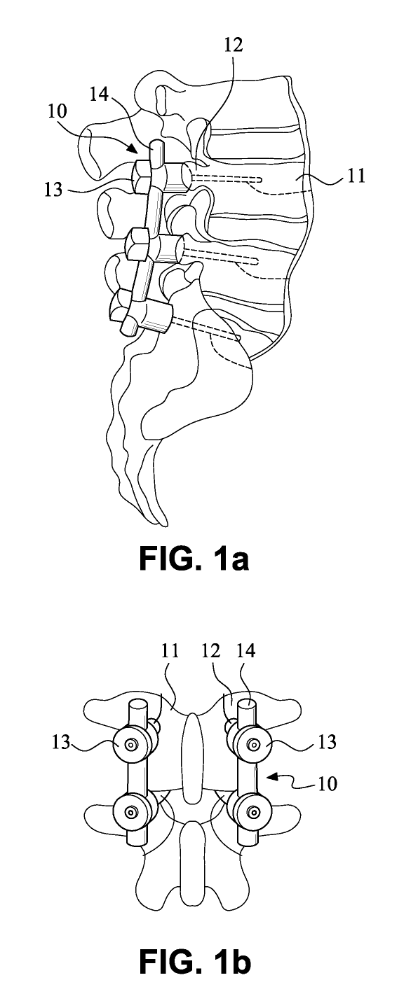

With reference to FIGS. 1a and 1b for schematic views of a conventional pedicle screw fixation system 10 and related surgical positions and methods, the conventional pedicle screw fixation system 10 implants a pedicle screw 13 at a position of a pedicle 12 on both sides of a vertebral body 11 separately, and then uses a vertical rod 14 to connect the pedicle screws 13 at different sections of a spine to achieve the effect of connecting and fixing the vertebral column and overcome the drawbacks and problems of the prior art.

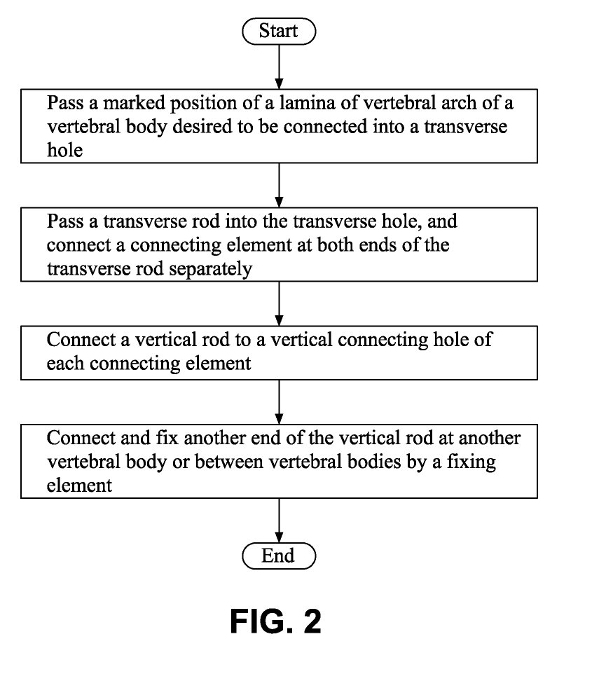

With reference to FIG. 2 for a flow chart of a method of connecting a transverse beam at a triangular position of a vertebral lamina in accordance with the present invention, the method is applied at a triangular intersection of a spinous process of a vertebral body and a lamina of vertebral arch, and the method comprises the following steps for a surgical operation procedure:
1. Pass a marked position of a lamina of vertebral arch of a vertebral body desired to be connected into a transverse hole.
2. Pass a transverse rod into the transverse hole, and connect a connecting element at both ends of the transverse rod separately.
3. Connect a vertical rod to a vertical connecting hole of each connecting element.
4. Connect and fix another end of the vertical rod at another vertebral body or between vertebral bodies by a fixing element.

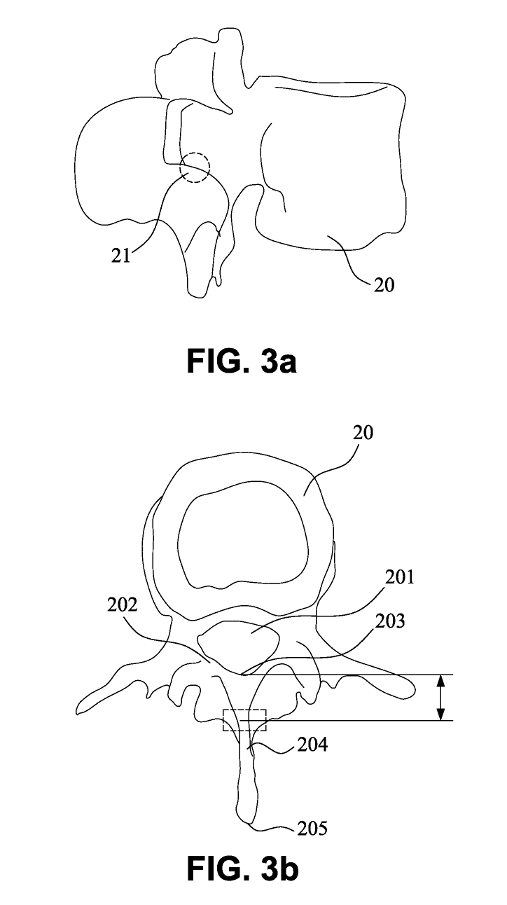

In FIGS. 3a and 3b, the transverse hole 21 of a marked lamina of vertebral arch of the vertebral body 20 is situated at a position of 7˜15 mm from a bottom vertex of the lamina of vertebral arch on a line connected from the bottom vertex 203 of the lamina of vertebral arch 202 at a curved position of a vertebral foramen 201 to a top vertex 205 of a spinous process 204

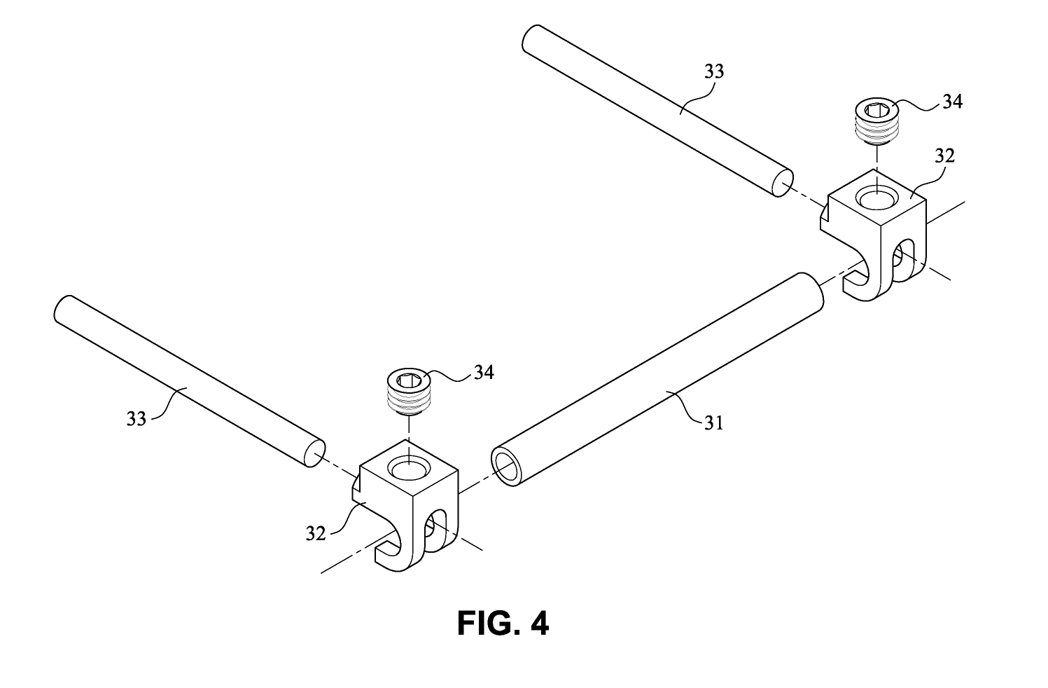

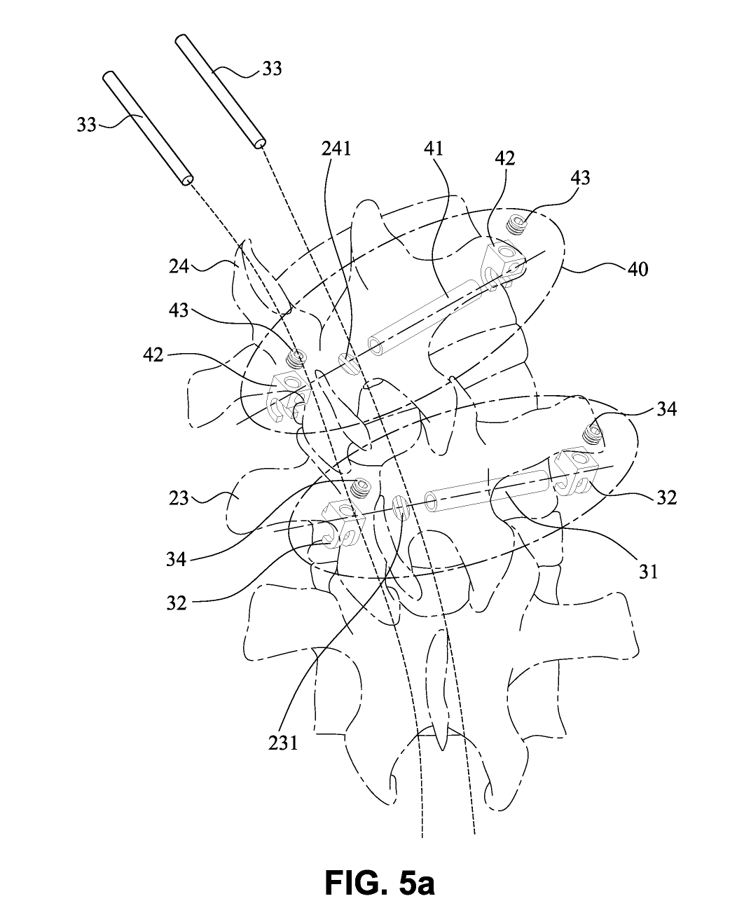

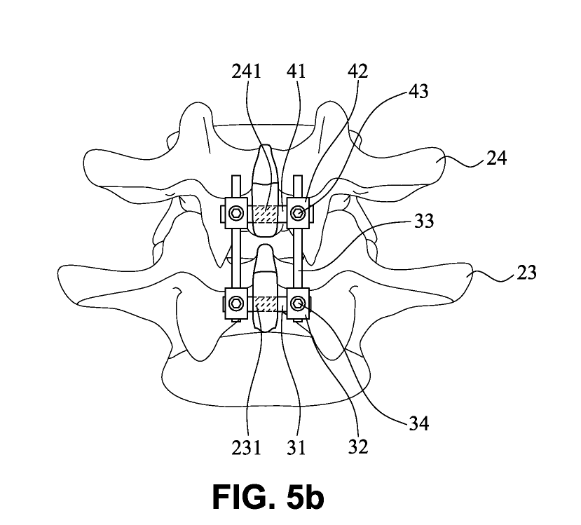

In FIG. 4, a device used in the method of connecting a transverse beam at a triangular position of a vertebral lamina comprises at least one transverse rod 31, two connecting elements 32, two vertical rods 33, two screws 34, and a fixing element or a set of fixing elements (as shown in FIG. 5) fixed and installed on a section of another vertebral body, wherein the transverse rod 31 is a transverse rod with an arc degree of curvature 311.

With reference to FIGS. 5a and 5b for the method of connecting a transverse beam at a triangular position of a vertebral lamina and its device installed at a section of a vertebral body required to be connected to a vertebral column, two vertebral bodies 23, 24 are shown, but the method and device of the invention are not limited for the application of connecting two vertebral bodies only, but they can be used for connecting a plurality of vertebral bodies at different positions as well.

In FIGS. 5a and 5b, a device used for the method of connecting a transverse beam at a triangular position of a vertebral lamina is installed at the vertebral bodies 23, and the device comprises a transverse rod 31 (or a curved transverse rod 311), two connecting elements 32, two vertical rods 33 (whose length is not limited to the length as shown in the figures only, but the length may be changed according to actual requirements), and the two screws 34 are installed securely at marked positions of transverse holes 231 of the vertebral body 23. The vertical rod 33 is connected by a fixing element 40 (wherein the fixing element enclosed by a dotted circle in the figure is another set of a combination of transverse rods and connecting elements).

The fixing element 40 (including a combination of transverse rods and connecting elements) comprises a transverse rod 41, two connecting elements 42, and two screws 43, and the fixing element 40 is installed to another vertebral body 24.

The marked position of the vertebral body 24 is also passed through a transverse hole 241, and the transverse rod 41 is passed into the transverse hole 241, and both ends of the transverse rod 41 are coupled to the connecting elements 42 respectively, and the connecting elements 42 are connected securely to the vertical rods 33 to form a “#”-shaped device for connecting two vertebral bodies (including upper and lower vertebral bodies).

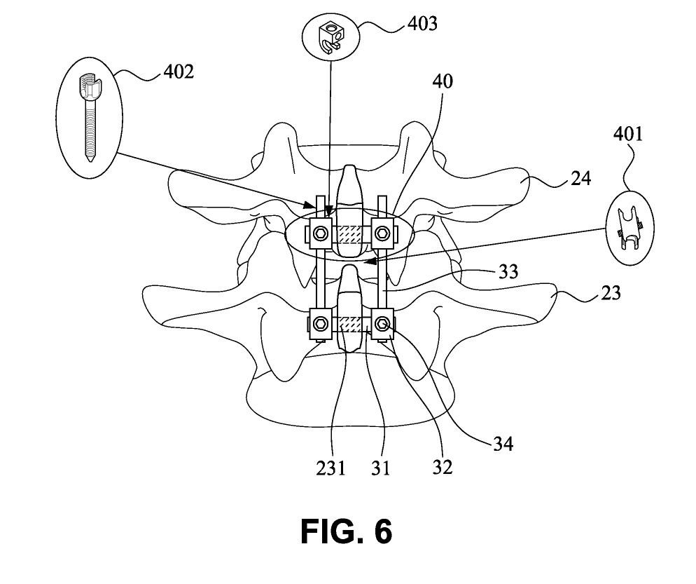

In addition to the aforementioned combination of transverse rods and connecting elements, the fixing element 40 (as shown in FIG. 6) of the device used in the method of connecting a transverse beam at a triangular position of a vertebral lamina is provided for fixing the vertical rods 33 to the following devices to complete a secured fixation of the vertebral column.

For example, an interspinous process fixation device 401 is installed between two adjacent spinous processes 23, 24, and both sides of the interspinous process fixation device 401 are coupled to the vertical rods 33 respectively.

Alternatively, a set of pedicle screws 402 are implanted and fixed to pedicle positions on both sides of another vertebral body 24, and the set of pedicle screws 402 are connected to the vertical rods 33 respectively.

Alternatively, a set of spinal hooks 403 are hooked at positions on both sides of another vertebral body 24 and coupled to the vertical rods 33 respectively.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method of connecting a transverse beam at a triangular position of a vertebral lamina, applied to a position below a spinous process of a vertebral body and above a triangular intersection position of a lamina of vertebral arch, and comprising the steps of;
passing a marked position of a lamina of vertebral arch of a vertebral body desired to be connected into a transverse hole;
passing a transverse rod into the transverse hole, and connecting a connecting element at both ends of the transverse rod separately;
connecting a vertical rod to a vertical connecting hole of each connecting element; and
connecting and fixing another end of the vertical rod at another vertebral body or between vertebral bodies by a fixing element.

2. The method of connecting a transverse beam at a triangular position of a vertebral lamina as recited in claim 1, wherein the marked transverse hole is transversally formed at a position of 7˜15 mm from the lamina of vertebral arch and on a line connected from a bottom vertex of the lamina of vertebral arch at a curved position to a top vertex of a spinous process.

3. The method of connecting a transverse beam at a triangular position of a vertebral lamina as recited in claim 1, wherein the transverse rod is a transverse rod with an arc degree of curvature.

4. The method of connecting a transverse beam at a triangular position of a vertebral lamina as recited in claim 1, wherein the fixing element is comprised of another transverse rod and a plurality of connecting elements, and the transverse rod is passed into a transverse hole at a marked position of another lamina of vertebral arch, and both ends of the transverse rod are coupled to another set of connecting elements, and the other set of connecting elements are coupled to the vertical rods.

5. The method of connecting a transverse beam at a triangular position of a vertebral lamina as recited in claim 1, wherein the fixing element is an interspinous process fixation device installed between two adjacent spinous processes, and both sides of the spinous process device are coupled to the vertical rods respectively.

6. The method of connecting a transverse beam at a triangular position of a vertebral lamina as recited in claim 1, wherein the fixing element is a set of pedicle screws implanted and fixed at pedicle positions on both sides of another vertebral body respectively, and the set of pedicle screws are coupled to the vertical rods.

7. The method of connecting a transverse beam at a triangular position of a vertebral lamina as recited in claim 1, wherein the fixing element is a set of spinal hooks hooked at positions on both sides of another vertebral body and coupled to the vertical rods.
